(19) United States
(12) Patent Application Publication
(10) Pub. No.: US 2026/0201006 A1
(43) Pub. Date: Jul. 16, 2026

(54) COACERVATE FORMULATIONS OF CXCR-ACTIVATING PEPTIDES FOR CONTROLLED RELEASE IN OPHTHALMIC AND TOPICAL TREATMENTS

(71) Applicants: University of Pittsburgh - Of the Commonwealth System of Higher Education, Pittsburgh, PA (US); The United States Government as represented by the Department of Veterans Affairs, Washington, DC (US); Cornell University, Ithaca, NY (US)

(72) Inventors: Alan H. Wells, Pittsburgh, PA (US); Shivalingappa Kottur Swamynathan, Tampa, FL (US); Yadong Wang, Ithaca, NY (US)

(73) Assignees: University of Pittsburgh - Of the Commonwealth System of Higher Education, Pittsburgh, PA (US); The United States Government as represented by the Department of Veterans Affairs, Washington, DC (US); Cornell University, Ithaca, NY (US)

(21) Appl. No.: 19/133,436

(22) PCT Filed: Nov. 29, 2023
(86) PCT No.: PCT/US2023/081556

§ 371 Date: May 28, 2025

Related U.S. Application Data
(60) Provisional application No. 63/385,588, filed on Nov. 30, 2022.

(51) Int. Cl. C07K 14/52; A61K 9/00; A61K 9/08; A61K 38/00; A61K 47/26; A61K 47/34

(52) U.S. Cl. CPC C07K 14/521; A61K 9/0048; A61K 9/08; A61K 47/26; A61K 47/34; A61K 38/00

(57) ABSTRACT

Controlled release coacervate formulations for slow, extended release of C-X-C chemokine receptor 3 (CXCR3) activating peptides are described. The controlled release compositions are designed for ophthalmic administration, such as by topical administration to the eye or by injection into the conjunctiva. Methods of treating dry eye disease and enhancing goblet cell density in the conjunctiva are also described.

### CROSS REFERENCE TO RELATED APPLICATIONS

This application claims the benefit of U.S. Provisional Application No. 63/385,588, filed Nov. 30, 2022, which is herein incorporated by reference in its entirety.

### FIELD

This disclosure concerns controlled, slow-release formulations of C-X-C chemokine receptor 3 (CXCR3) activating peptides for the treatment of dry eye disease and promotion of goblet cell survival.

### INCORPORATION OF ELECTRONIC SEQUENCE LISTING

The electronic sequence listing, submitted herewith as an XML file named 8123-109165-02.xml (11,828 bytes), created on Nov. 8, 2023, is herein incorporated by reference in its entirety.

### BACKGROUND

Dry eye disease (DED), also known as keratoconjunctivitis sicca (KCS), is a syndrome with a number of different causes. The basal phenotype of insufficient tear coverage of the sclera is often treated palliatively with physical replacement of tears. The only treatments that target a cause of DED are those that ameliorate auto-immune disease related inflammation; however, these immunosuppressive agents are germane against only about 1 in 10 cases of DED. This leaves the vast majority of cases with only temporary symptomatic relief.

Regardless of the mechanistic cause, the tissue manifestations of DED converge on damage to the sclera of the eye. Once this is compromised, the goblet cells malfunction and are eventually lost due to non-specific inflammatory damage. This leads to a decrease in the mucin layer of the tears, with resultant inability of the aqueous layer to adhere to the scleral surface. Once the goblet cell mucin is insufficient, additional aqueous components are temporary, leading to either frequent administration or even the paradoxical watery/teary dry eye symptoms.

Thus, a need exists for effective treatments for DED, particularly treatments that can be self-administered (e.g., as a topical formulation) and do not require multiple administrations per day to maintain symptom relief.

### SUMMARY

Controlled release formulations for slow, extended release of C-X-C chemokine receptor 3 (CXCR3) activating peptides are described. The controlled release compositions are designed for ophthalmic administration and can be used, for example, to preserve goblet cells in the conjunctiva and treat dry eye disease (DED).

Provided herein are compositions that include one or more CXCR3 activating peptides in a controlled release formulation for ophthalmic administration. In some aspects, the controlled release formulation includes a coacervate. In particular examples, the coacervate includes poly(ethylene arginyl aspartate diglyceride) (PEAD) and heparin.

In some aspects, the one or more CXCR3-activating peptides are biologically active fragments or variants of a C-X-C chemokine ligand 4 (CXCL4), CXCL9, CXCL10 or CXCL11 protein. In some examples, the CXCR3 activating peptide is a 19 to 23 amino acid fragment of CXCL10, such as the CXCL10p peptide set forth as SEQ ID NO: 5.

Also provided herein are methods of treating DED in a subject, and methods of increasing goblet cell density in a subject. In some aspects, the methods include administering to the subject a therapeutically effective amount of a composition disclosed herein. In some examples, the composition is administered topically as an eye drop or eye cream. In other examples, the composition is administered by injection into the conjunctiva. The composition can be administered to the subject, such as a subject with DED, at appropriate intervals, such as once per week, twice per week, three times per week, once per day or twice per day.

The foregoing and other features of this disclosure will become more apparent from the following detailed description of several aspects which proceeds with reference to the accompanying figures.

### BRIEF DESCRIPTION OF THE DRAWINGS

FIG. 1: Increase in tear break-up time (TBUT) upon treatment of rabbits. Rabbits (8 per group) were treated with the CXCL10p peptide (SEQ ID NO: 5) either weekly in a subconjunctival injection formulation (ConjInj), twice per day in saline eye drops (EyeDrop), or twice per day in a coacervate formulation (SRPeptide). Rabbits administered saline eye drops twice per day were included as controls. On days 2, 7, 9, 14, 16, and 21, eyes were assessed for TBUT and Schirmer tear test (STT). Shown in the figure are averages, with all treatments groups being statistically greater than saline control by day 7 (p<0.05 using t-Test comparisons).

FIG. 2: Decreased TNFα production in the presence of CXCL10p peptide. Human conjunctival epithelial (HCjE) cells were exposed to TNFα (5 ng/ml) in the presence or absence of CXCL10p peptide (200 ng/ml) for 4 hours prior to harvesting and qRT-PCR. CXCL10p peptide reduced TNFα mRNA levels by approximately one quarter. Shown is the mean±s.d. of three experiments each in triplicate; P<0.01 comparing TNFα+IP10p to TNFα.

### SEQUENCE LISTING

The amino acid sequences listed in the accompanying sequence listing are shown using standard single letter code for amino acids, as defined in 37 C.F.R. 1.822. In the accompanying sequence listing:

SEQ ID NO: 1 is an exemplary amino acid sequence of human CXCL4 (GENBANK™ Accession No. NP_002610.1).

SEQ ID NO: 2 is an exemplary amino acid sequence of human CXCL9 (GENBANK™ Accession No. NP_002407.1).

SEQ ID NO: 3 is an exemplary amino acid sequence of human CXCL10 (GENBANK™ Accession No. NP_001556.2).

SEQ ID NO: 4 is an exemplary amino acid sequence of human CXCL11 (GENBANK™ Accession No. NP_005400.1).

SEQ ID NOs: 5 is the amino acid sequence of the CXCL10p peptide.

SEQ ID NOs: 6-9 are amino acid sequences of CXCR3 activating peptides derived from CXCL10.

SEQ ID NOs: 10-12 are amino acid sequences of CXCR3 activating peptides derived from CXCL4.

### DETAILED DESCRIPTION

### I. Introduction

Dry eye disease (DED) is the most prevalent eye condition, afflicting approximately 16 million Americans and up to 25% of people in the developed world. There are many causes of DED, including environmental factors, aging, medications, and trauma caused by toxins or eye surgery. DED results in damage to the cornea and destruction of goblet cells, which are responsible for secreting mucins that promote ocular surface wettability and maintain tear film. Currently, there are no approved therapeutics for treating the underlying causes of DED (such as the loss of goblet cells). Thus, subjects suffering from DED can only treat the symptoms of DED, such as by administering artificial tears multiple times per day.

The present disclosure meets the need for an effective DED therapeutic by providing a controlled (slow) release coacervate formulation of CXCR3 activating peptide, which promotes goblet cell survival and expansion, while also improving tear film stability. The disclosed compositions not only provide immediate symptomatic relief, but they also minimize the number of administrations required to maintain symptom relief.

### II. Abbreviations

BID twice daily
CXCR3 C-X-C motif chemokine receptor 3
CXCL4 C-X-C motif chemokine ligand 4
CXCL9 C-X-C motif chemokine ligand 9
CXCL10 C-X-C motif chemokine ligand 10
CXCL11 C-X-C motif chemokine ligand 11
DED dry eye disease
HCjE human conjunctival epithelial
KCS keratoconjunctivitis sicca
MUC5AC mucin 5AC
OD right eye
OS left eye
OU both eyes
PAS periodic acid-Schiff's
PEAD poly(ethylene arginyl aspartate diglyceride)
STT Schirmer tear test
TBP TATA-box binding protein
TBUT tear break-up time

### III. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Administration: The introduction of a composition (such as a peptide or controlled delivery composition disclosed herein) into a subject by a chosen route. Exemplary routes of administration include, but are not limited to, ophthalmic administration, injection (such as intraocular, intravitreal, conjunctival, subcutaneous, intramuscular, intradermal, intraperitoneal, and intravenous), oral, intraductal, sublingual, transdermal, intranasal, topical, inhalation, and via a medical implant. As used herein, ophthalmic administration includes any route of administration that brings the composition to be administered into contact with a subject's eye. In some examples, ophthalmic administration includes topical administration, such as by administration of an eye drop, eye spray or eye cream, or a depot placed under the eyelid. In other examples, ophthalmic administration includes injection into the conjunctiva.

Biologically active fragment or variant: Any fragment or variant of a protein that retains an activity of the protein. In the context of the present disclosure, a biologically active fragment or variant of a peptide that activates CXCR3 is one that retains the ability to bind CXCR3 and/or to increase tear production in the eye and/or to prevent the loss of goblet cells in the eye, and/or to alleviate one or more symptoms of dry eye disease. In some aspects, the peptide variant comprises no more than 1, no more than 2, or nor more than 3 amino acid substitutions compared to a native peptide sequence (such as a wild-type CXCL4, CXCL9, CXCL10, or CXCL11 protein set forth herein as SEQ ID NOs: 1-4, respectively). The substitutions can be conservative or non-conservative substitutions. In some examples, the substitutions are conservative. In some aspects, the biologically active fragment is a portion of a native protein (such as a wild-type CXCL4, CXCL9, CXCL10, or CXCL11 protein set forth herein as SEQ ID NOs: 1-4, respectively) that is about 12 to about 30 amino acids in length, such as about 15 to about 25 amino acids in length or about 19 to about 23 amino acids in length. In particular examples, the biologically active fragment is 12, 13, 14, 15, 16, 17, 18, 19, 20, 21, 22, 23, 24, 25, 26, 27, 28, 29 or 30 amino acids in length.

Chemical modification (of a peptide): A modification that alters one or more properties of a peptide. In some examples, the modification increases stability, solubility, hydrophobicity and/or charge of the peptide. For example, the peptide can be modified by conjugation to a large inert molecule. In some examples, the modification is conjugation of the peptide to polyethylene glycol (PEG), a largely inert matrix protein such as collagen or gelatin, or dextran (see, for example, Mehvar, “Dextrans for targeted and sustained delivery of therapeutic and imaging agents,” J Control Release 69(1):1-25, 2000).

Coacervate: Spherical aggregates of colloidal droplets held together (and apart from their surrounding liquid environment) by hydrophobic forces. Coacervate droplets are generally about 0.1 to about 100 μm in diameter. Coacervates typically aggregate over time to form a bulk phase separation from the aqueous compartment. Coacervates can be used as controlled delivery vehicles for small molecules and protein therapeutics. Advantageous features of a coacervate for drug delivery include their high loading capacity and ability to self-assemble in aqueous media (see, e.g., Johnson and Wang, Expert Opin Drug Deliv 11(12):1829-1832, 2014). In some aspects herein, the coacervate includes a positively charged synthetic biodegradable poly(ethylene arginyl aspartate diglyceride) (PEAD) and a negatively charged heparin, which forms a 3-dimensional coacervate that envelopes around the protein cargo to be administered. Other coacervates can be used, such as those described in U.S. Patent Application Publication Nos. 2019/0117780 and 2021/0290770.

Conjunctiva: The mucous membrane that lines the inner surface of the eyelid and the outer surface of the eye.

Conservative variants: “Conservative” amino acid substitutions are those substitutions that do not substantially affect or decrease an activity or antigenicity of a protein or peptide. For example, a peptide disclosed herein can include at most about 1, at most about 2, at most about 3, at most about 4 or at most about 5 conservative substitutions (such as 1, 2, 3, 4, or 5 conservative substitutions, and retain biological activity, such as the ability to bind CXCR3. Specific, non-limiting examples of a conservative substitution include the following examples:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variant also includes the use of a substituted amino acid in place of an unsubstituted parent amino acid. Non-conservative substitutions are those that reduce an activity or antigenicity.

Controlled release formulation: A formulation in which the rate of release of the component(s) (e.g., peptides, drugs and/or therapeutic agents) is regulated, for example to delay or extend release of the component(s) over time.

C-X-C motif chemokine ligand 4 (CXCL4): A small cytokine belonging to the CXC chemokine family. CXCL4 is also known as platelet factor 4 (PF4). The CXCL4 protein is released from the alpha-granules of activated platelets and binds with high affinity to heparin. Its major physiologic role is neutralization of heparin-like molecules on the endothelial surface of blood vessels, thereby inhibiting local antithrombin III activity and promoting coagulation. As a strong chemoattractant for neutrophils and fibroblasts, CXCL4 is believed to play a role in inflammation and wound repair. CXCL4 is known to bind the B isoform of CXCR3 (CXCR3-B). Sequences for CXCL4 are publicly available (see, for example, GENBANK™ Gene ID 5196). An exemplary human CXCL4 amino acid sequence is set forth herein as SEQ ID NO: 1.

C-X-C motif chemokine ligand 9 (CXCL9): A member of the CXC chemokine family. The CXCL9 protein is thought to be involved in T cell trafficking. CXCL9 binds to CXCR3 and is a chemoattractant for lymphocytes, but not for neutrophils. Sequences for CXCL4 are publicly available (see, for example, GENBANK™ Gene ID 4283). An exemplary human CXCL4 sequence is set forth herein as SEQ ID NO: 2.

C-X-C motif chemokine ligand 10 (CXCL10): A chemokine of the CXC subfamily and a ligand for the receptor CXCR3. CXCL10 is also known as interferon-γ-inducible 10 kDa protein (IP-10). Binding of this protein to CXCR3 results in pleiotropic effects, including stimulation of monocytes, natural killer and T-cell migration, modulation of adhesion molecule expression, and inhibition of vessel formation. CXCL10 sequences are publicly available, such as through GENBANK™ (see, for example, Gene ID 3627 for human CXCL10 sequences; see also GENBANK™ Accession No. P02778). An exemplary human CXCL10 sequence is set forth herein as SEQ ID NO: 3.

C-X-C motif chemokine ligand 11 (CXCL11): A chemokine of the CXC subfamily and a ligand for the receptor CXCR3. The CXCL11 protein induces a chemotactic response in activated T-cells and is the dominant ligand for CXCR3. The gene encoding this protein contains 4 exons and at least three polyadenylation signals which may reflect cell-specific regulation of expression. IFN-γ is a potent inducer of transcription of this gene. Sequences for CXCL11 are publicly available (see, for example, GENBANK™ Gene ID 6373). An exemplary human CXCL11 sequence is set forth herein as SEQ ID NO: 4.

C-X-C motif chemokine receptor 3 (CXCR3): A G protein-coupled receptor with selectivity for four chemokines, CXCL4/PF4 (platelet factor 4), CXCL9/Mig (monokine induced by interferon-γ), CXCL10/IP-10 (interferon-γ-inducible 10 kDa protein) and CXCL11/1-TAC (interferon-inducible T cell a-chemoattractant). Binding of chemokines to this protein induces cellular responses that are involved in leukocyte trafficking, most notably integrin activation, cytoskeletal changes, and chemotactic migration. Alternatively spliced transcript variants encoding different isoforms have been found for this gene. One of the isoforms (CXCR3-B) shows high affinity binding to chemokine CXCL4.

C-X-C motif chemokine receptor 3 (CXCR3) activating peptide: A peptide that selectively interacts with (such as binds) CXCR3 and promotes, enhances, or increases one or more functions or activities of CXCR3, such as promoting goblet cell survival and/or expansion, and/or increasing goblet cell density. In some aspects, the CXCR3 activating peptide is a fragment and/or variant of a CXCR3 ligand (e.g., CXCL4, CXCL9, CXCL10, or CXCL11). In particular examples, the CXCR3 activating peptide is a fragment/variant of CXCL10, such as the CXCL10p peptide set forth herein as SEQ ID NO: 5, or a peptide set forth herein as any one of SEQ ID NOs: 6-9. In other particular examples, the CXCR3 activating peptide is a fragment/variant of CXCL4, such as a peptide set forth herein as any one of SEQ ID NOs: 10-12.

Dry eye disease (DED): A multifactorial disease of the tears and ocular surface that results in discomfort, visual disturbance, and tear film instability. Dry eye disease is generally caused by either decreased tear production or increased tear film evaporation. DED is also known as keratoconjunctivitis sicca (KCS) or keratitis sicca. A number of different factors or conditions are associated with the development of DED, including age (eye dryness increases with age), gender (women are more likely to develop dry eye from hormonal changes associated with pregnancy, menopause or the use of oral contraceptives), use of medications that inhibit tear production, medical conditions associated with dry eyes and/or lacrimal gland dysfunction (e.g. rheumatoid arthritis, Sjögren's syndrome, diabetes and thyroid disorders), environmental conditions that increase tear evaporation (e.g., exposure to smoke, wind and dry climates), contact lens use, and refractive eye surgery (such as LASIK). In the context of the present disclosure, DED can be caused by any one or any combination of diseases, conditions, or other factors.

Goblet cells: Glandular epithelial cells that secrete mucin. Goblet cells are found in the conjunctiva and in the epithelial lining of many organs, such as in the intestinal and respiratory tracts. Goblet cells are the primary source of tear mucus.

Heparin: A member of the glycosaminoglycan family of carbohydrates that is comprised of a variably sulfated repeating disaccharide unit. In the context of the present disclosure, the heparin is a clinical grade heparin (having sufficient purity for use in humans) from any source. Full-length heparin or shorter versions of heparin, including heparin fragments, are contemplated for use in the coacervate formulations disclosed herein.

Increasing goblet cell density: Increasing the number of goblet cells within in a particular tissue (or particular section of tissue), e.g., in the conjunctiva of a subject. Goblet cell density can increase, for example, by replenishing goblet cells that have been lost due to a particular disease or condition. Goblet cell density can also be increased by preventing the loss of goblet cells, such as the loss of goblet cells that would occur as the result of a medical condition (such as DED) in the absence of treatment.

Non-canonical amino acid: An amino acid that is not one of the twenty amino acids encoded directly by triplet codons in the genetic code. Non-canonical amino acids are also referred to as “non-standard” amino acids.

Peptide or polypeptide: A polymer in which the monomers are amino acid residues which are joined together through amide bonds. When the amino acids are alpha-amino acids, either the L-optical isomer or the D-optical isomer can be used, the L-isomers being preferred. The terms “polypeptide,” “peptide,” or “protein” as used herein are intended to encompass any amino acid sequence and include modified sequences such as glycoproteins. The terms “polypeptide” and “peptide” are specifically intended to cover naturally occurring proteins, as well as those which are recombinantly or synthetically produced. In some aspects, a peptide is between 10 and 200 amino acids in length, including 10 to 100, 10 to 50, 10 to 30, 15 to 50, 15 to 30 or 18 to 25 amino acids in length. In particular examples, the peptide is about 12 to about 30, about 15 to about 25, about 19 to about 23, or about 21 or about 22 amino acids in length. A “residue” refers to an amino acid or amino acid mimetic incorporated in a polypeptide by an amide bond or amide bond mimetic.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the peptides and controlled release formulations disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. For topical application to the eye, agents can be mixed, for example, with artificial tears and other emulsions.

Poly(ethylene arginyl aspartate diglyceride) (PEAD): A biocompatible polycation made from glycerol, glutamic acid and arginine. Methods of synthesizing PEAD and PEAD-heparin are known (see, e.g., U.S. Pat. No. 9,023,972 and U.S. Publication No. 2018/0360976).

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a particular polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. In addition, Altschul et al., Nature Genet. 6:119, 1994, presents a detailed consideration of sequence alignment methods and homology calculations.

The NCBI Basic Local Alignment Search Tool (BLAST) (Altschul et al, J. Mol Biot. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least about 75%, for example at least about 80%, 90%, 95%, 96%, 97%, 98% or 99% sequence identity counted over the full length alignment with the amino acid sequence of the polypeptide using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Subject: Living multi-cellular vertebrate organisms, a category that includes both human and veterinary subjects, including human and non-human mammals. In some examples, the subject is a human subject with dry eye disease.

Therapeutically effective amount: A quantity of a specified agent (such as a CXCR3 activating peptide) sufficient to achieve a desired effect in a subject, cell or culture being treated with that agent. In some examples, the therapeutically effective amount is an amount necessary to reduce the symptoms of dry eye disease by at least 10%, at least 20%, at least 30%, at least 40%, at least 50%, at least 60%, at least 70%, at least 80%, at least 90%, or at least 100% or more, relative to dry eye in the absence of treatment. In other examples, the therapeutically effective amount is an amount necessary to increase goblet cell density in the eye by at least 10%, at least 20%, at least 30%, at least 40%, at least 50%, at least 60%, at least 70%, at least 80%, at least 90%, or at least 100% or more, relative to goblet cell density in the absence of treatment. In other examples, the therapeutically effective amount is an amount necessary to increase tear production by at least 10%, at least 20%, at least 30%, at least 40%, at least 50%, at least 60%, at least 70%, at least 80%, at least 90%, or at least 100% or more, relative to tear production in the absence of treatment. In yet other examples, the therapeutically effective amount is an amount necessary to increase tear break-up time (TBUT) by at least 10%, at least 20%, at least 30%, at least 40%, at least 50%, at least 60%, at least 70%, at least 80%, at least 90%, or at least 100% or more, relative to TBUT in the absence of treatment.

### IV. Compositions and Methods for Treating Dry Eye Disease

The present disclosure describes controlled release, coacervate formulations of CXCR3 activating peptides for ophthalmic administration. The controlled (slow) release compositions can be used, for example, to preserve goblet cells (or increase goblet cell density) in the conjunctiva and treat dry eye disease (DED).

Provided herein are compositions that include one or more CXCR3 activating peptides in a controlled release formulation for ophthalmic administration. In some aspects, the controlled release formulation includes a coacervate. In some examples, the coacervate includes poly(ethylene arginyl aspartate diglyceride) (PEAD) and heparin. In particular examples, the heparin is full-length heparin. In other examples, the heparin includes heparin fragments.

The CXCR3 activating peptide can be any peptide ligand that binds to and promotes, enhances or increases at least one biological activity or function of CXCR3. For example, the CXCR3 activity can be, but is not limited to, promoting goblet cell survival, increasing goblet cell expansion and/or increasing goblet cell density (such as in the conjunctiva). In some aspects, the CXCR3 activating peptide is a fragment and/or variant of a CXCR3 ligand, such as CXCL4, CXCL9, CXCL10, or CXCL11 (for example, human CXCL4, CXCL9, CXCL10, or CXCL11). In some examples, the amino acid sequence of the CXCR3 activating peptide is at least 80%, at least 85%, at least 90%, at least 95%, at least 96%, at least 97%, at least 98%, or at least 99% identical to a fragment (such as a fragment of 12 to 30 amino acids in length) of SEQ ID NO: 1, SEQ ID NO: 2, SEQ ID NO: 3 or SEQ ID NO: 5.

In particular examples, the CXCR3 activating peptide is a fragment/variant of CXCL10, such as the CXCL10p peptide set forth herein as SEQ ID NO: 5, or a CXCL10-derived peptide set forth herein as any one of SEQ ID NOs: 6-9. In some examples, the amino acid sequence of the CXCR3 activating peptide is at least 80%, at least 85%, at least 90%, at least 95%, at least 96%, at least 97%, at least 98%, or at least 99% identical to any one of SEQ ID NOs: 5-9. In specific examples, the amino acid sequence of the CXCR3 activating peptide comprises or consist of any one of SEQ ID NOs: 5-9. In some examples, the amino acid sequence of the CXCR3 activating peptide comprises SEQ ID NO: 5, SEQ ID NO: 6, SEQ ID NO: 7, SEQ ID NO: 8 or SEQ ID NO: 9 with no more than 5, no more than 4, no more than 3, no more than 2 or no more than 1 amino acid substitution, such as a conservative amino acid substitution.

In other particular examples, the CXCR3 activating peptide is a fragment/variant of CXCL4, such as a CXCL4-derived peptide set forth herein as any one of SEQ ID NOs: 10-12. In some examples, the amino acid sequence of the CXCR3 activating peptide is at least 80%, at least 85%, at least 90%, at least 95%, at least 96%, at least 97%, at least 98%, or at least 99% identical to any one of SEQ ID NOs: 10-12. In specific examples, the amino acid sequence of the CXCR3 activating peptide comprises or consist of any one of SEQ ID NOs: 10-12. In some examples, the amino acid sequence of the CXCR3 activating peptide comprises SEQ ID NO: 10, SEQ ID NO: 11 or SEQ ID NO: 12 with no more than 5, no more than 4, no more than 3, no more than 2 or no more than 1 amino acid substitution, such as a conservative amino acid substitution.

In some aspects, the CXCR3 activating peptide is about 12 to about 30 amino acids in length. In some examples, the CXCR3 activating peptide is about 15 to about 25 amino acids in length, or about 19 to about 23 amino acids in length. In specific examples, the CXCR3 activating peptide is 12, 13, 14, 15, 16, 17, 18, 19, 20, 21, 22, 23, 24, 25, 26, 27, 28, 29 or 30 amino acids in length. In particular examples, the CXCR3 activating peptide is 20, 21 or 22 amino acids in length.

In some aspects, the composition includes a single CXCR3 activating peptide. In some examples, the single CXCR3 activating peptide has an amino acid sequence comprising or consisting of PESKAIKNLLKAVHKEMSKRSP (SEQ ID NO: 5; CXCL10p) or ESKAIKNLLKAVHKEMSKRS (residues 2-21 of SEQ ID NO: 5).

In other aspects, the composition includes at least two CXCR3 activating peptides. In some examples, the amino acid sequence of one of the peptides comprises or consists of SEQ ID NO: 5, or residues 2-21 of SEQ ID NO: 5. In particular examples, the amino acid sequence of one of the peptides comprises or consists of SEQ ID NO: 5, or residues 2-21 of SEQ ID NO: 5, and the amino acid sequence of a second peptide comprises or consists of any one of SEQ ID NOs: 6-12.

In specific examples, the amino acid sequence of the CXCR3 activating peptide (or one of the CXCR3 activating peptides) comprises SEQ ID NO: 5 (or residues 2-21 of SEQ ID NO: 5) and the methionine at residue 17 (with reference to SEQ ID NO: 5) is in the reduced form. In other examples, the amino acid sequence of the CXCR3 activating peptide (or one of the CXCR3 activating peptides) comprises SEQ ID NO: 5 (or residues 2-21 of SEQ ID NO: 5) and the methionine at residue 17 (with reference to SEQ ID NO: 5) is in the oxidized form.

In some aspects, the CXCR3 activating peptide has at least one chemical modification. Generally, the modification alters one or more properties of a peptide. In some examples, the modification increases stability, half-life, solubility, hydrophobicity and/or charge of the peptide and/or inhibits degradation of the peptide. For example, the peptide can be modified by conjugation to a large inert molecule. In particular examples, the modification is conjugation of the peptide to polyethylene glycol (PEG) or dextran. In some examples, the at least one modification comprises a modification at the N-terminus of the peptide, a modification at the C-terminus of the peptide, or both. In specific non-limiting examples, the modification at the N-terminus comprises formylation, acetylation, propionylation, pyroglutamate formation, myristoylation, palmitylation, S-palmitoylation, mono-methylation, di-methylation, tri-methylation, or any combination thereof. In other specific non-limiting examples, the modification at the C-terminus comprises methylation, alpha-amidation, or a combination thereof. In other aspects, the at least one chemical modification comprises a non-standard peptide linkage.

In some aspects, the CXCR3 activating peptide includes at least one D-amino acid. In some examples, the CXCR3 activating peptide includes multiple D-amino acids, such as at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or at least 21 D-amino acids.

In some aspects, the CXCR3 activating peptide includes at least one non-canonical amino acid. In some examples, the at least one non-canonical amino acid is a modified non-canonical amino acid. The peptide can include a modified non-canonical amino acid at the N-terminus of the peptide, at the C-terminus of the peptide, or both. In specific non-limiting examples, the peptide includes a modified non-canonical amino acid at the N-terminus and the modification comprises formylation, acetylation, propionylation, pyroglutamate formation, myristoylation, palmitylation, S-palmitoylation, mono-methylation, di-methylation, tri-methylation, or any combination thereof. In other specific non-limiting examples, the peptide comprises a modified non-canonical amino acid at the C-terminus and the modification comprises methylation, alpha-amidation, or a combination thereof.

In some aspects, the at least one non-canonical amino acid is a methylated amino acid, an amino acid conjugated to a polyethylene glycol polymer, an amino acid conjugated to biotin, an amino acid conjugated to fluorescein isothiocyanate (FITC), an amino acid conjugated to a carrier protein, an amino acid labelled with a radioactive isotope, or any combination thereof. In some examples, the methylated amino acid is a mono-methylated amino acid, di-methylated amino acid, or tri-methylated amino acid. In some examples, the carrier protein is bovine serum albumin, ovalbumin, or keyhole limpet hemocyanin. In some examples, the radioactive isotope is ²H, ⁵N, ¹³C, or both ¹⁵N and ¹³C

In one non-limiting example, provided is a composition that includes a CXCR3 activating peptide in a controlled release formulation for ophthalmic administration, wherein the amino acid sequence of the CXCR3 activating peptide comprises PESKAIKNLLKAVHKEMSKRSP (SEQ ID NO: 5); and the controlled release formulation comprises a coacervate comprising poly(ethylene arginyl aspartate diglyceride) (PEAD) and heparin.

Also provided herein are methods of treating DED in a subject. Further provided herein are methods of increasing goblet cell density in a subject. In some aspects, the methods include administering to the subject a therapeutically effective amount of a composition disclosed herein. In some examples, the composition is administered topically as an eye drop or eye cream. In other examples, the composition is administered by injection into the conjunctiva.

In some aspects, the methods further include selecting a subject with DED. The DED in the subject can be caused by any disease, condition, or disorder (or combination thereof) that lead to DED. For example, DED may be the result of increased age, hormonal changes, the use of medications that inhibit tear production, medical conditions associated with dry eyes and/or lacrimal gland dysfunction such as keratoconjunctivitis sicca, exposure to environmental conditions that increase tear evaporation, the use of contact lenses, or ophthalmic surgery, such as refractive eye surgery. In some examples of the disclosed methods, the subject has rheumatoid arthritis, Sjögren's syndrome, diabetes, or a thyroid disorder. In other examples, the subject has previously had refractive eye surgery, or has cicatricial changes that cause exposure of the cornea, as in cicatricial entropion.

The composition can be administered at any suitable interval, such as at a frequency that provides symptomatic relief of DED and/or that maintains or increases goblet cell density. In some aspects, the composition is administered once per week, twice per week, three times per week, once per day or twice per day. In some examples, the composition is administered more frequently at the start of treatment, followed by less frequent administration (such as to maintain symptom relief and/or to maintain the density of goblet cells in the conjunctiva). In specific examples, the composition is administered daily for about one week, followed by administration 1 to 3 times per week.

In some aspects, the concentration of the CXCR3 activating peptide in the composition is about 5 μg to about 500 μg per 50 μl of the coacervate, such as about 10 μg to about 400 μg, about 20 μg to about 300 μg, about 30 μg to about 200 μg, about 40 μg to about 150 μg, or about 50 μg to about 100 μg per 50 μl of the coacervate.

In some aspects, the composition is administered to the subject at a volume sufficient to deliver about 10 μg to about 1 mg of the CXCR3 activating peptide, such as about 20 μg to about 750 μg, about 30 μg to about 500 μg, about 40 μg to about 250 μg, about 50 μg to about 150 μg, or about 75 μg to about 100 μg of the CXCR3 activating peptide.

### V. CXCR3 Activating Peptide Sequences and Variants

In some aspects of the present disclosure, the controlled release coacervate compositions include a peptide derived from a CXCL10, CXCL4, CXCL9 or CXCL11 protein, or a biologically active peptide fragment or variant thereof.

In particular aspects, the CXCR3 peptide is a fragment or variant of CXCL10. Sequences for CXCL10 proteins from a variety of different species are publicly accessible, such as through the GENBANK™ database. For example, CXCL10 sequences are known for at least the following species: human (see GENBANK™ Gene ID 3627), mouse (Gene ID 15945), rat (Gene ID 24592), pig (Gene ID 494019), chimpanzee (Gene ID 461242), dog (Gene ID 478432), cow (Gene ID 615107), macaque (Gene ID 574243), horse (Gene ID 100050993) and sheep (Gene ID 44297).

In some aspects of the compositions and methods disclosed herein, the CXCL10 protein is human CXCL10, or a biologically active fragment or variant thereof. Exemplary CXCL10 protein and peptide sequences are provided below.

Human CXCL10
(GENBANK ™ Accession No. NP_001556.2):
(SEQ ID NO: 3)
MNQTAILICCLIFLTLSGIQGVPLSRTVRCTCISISNQPV
NPRSLEKLEIIPASQFCPRVEIIATMKKKGEKRCLNPESK
AIKNLLKAVSKERSKRSP
CXCL10 peptide fragments/variants:
(SEQ ID NO: 5; CXCL10p)
PESKAIKNLLKAVHKEMSKRSP
(SEQ ID NO: 6)
PESKAIKNLLKAVSKEMSKRSP
(SEQ ID NO: 7)
ESKAIKNLLKAVSKERSKRSP
(SEQ ID NO: 8)
PESKAIKNLLKAVSKERSKRSP
(SEQ ID NO: 9)
ESKAIKNLLKAVSKEMSKRSP

In some aspects, the CXCL10 peptide is derived from a human CXCL10 sequence that is at least 85%, at least 90%, at least 95%, at least 96%, at least 97%, at least 98 or at least 99% identical to SEQ ID NO: 3. In some examples, the CXCL10 peptide is no more than 30 amino acids in length, such as a biologically active fragment of CXCL10 that is 12 to 30, 15 to 25, or 19 to 23 amino acids in length. In specific examples, the CXCL10 peptide is a human CXCL10 fragment or variant that is 19, 20, 21, 22 or 23 amino acids in length. In particular examples, the amino acid sequence of the CXCL10 peptide is at least 85%, at least 90%, at least 95%, at least 96%, at least 97%, at least 98 or at least 99% identical to SEQ ID NO: 5, SEQ ID NO: 6, SEQ ID NO: 7, SEQ ID NO: 8 or SEQ ID NO: 9. In specific non-limiting examples, the amino acid sequence of the CXCL10 peptide comprises or consists of SEQ ID NO: 5, SEQ ID NO: 6, SEQ ID NO: 7, SEQ ID NO: 8 or SEQ ID NO: 9.

In other particular aspects, the CXCR3 peptide is a fragment or variant of CXCL4. Sequences for CXCL4 proteins from a variety of different species are publicly accessible, such as through the GENBANK™ database. For example, CXCL4 sequences are known for at least the following species: human (see GENBANK™ Gene ID 5196), mouse (Gene ID 56744), rat (Gene ID 360918), chimpanzee (Gene ID 740477), cow (Gene ID 507790) and macaque (Gene ID 703451).

In some aspects of the compositions and methods disclosed herein, the CXCL4 protein is human CXCL4, or a biologically active fragment thereof. Exemplary CXCL4 protein and peptide sequences are provided below.

Human CXCL4 protein
(GENBANK ™ Accession No. NP_002610.1):
(SEQ ID NO: 1)
MSSAAGFCASRPGLLFLGLLLLPLVVAFASAEAEEDGDLQCL
CVKTTSQVRPRHITSLEVIKAGPHCPTAQLIATLKNGRKICL
DLQAPLYKKIIKKLLES
CXCL4 peptides:
(SEQ ID NO: 10)
DLQCLCVKTTSQVRPRHITSLEVIKAGPH
(SEQ ID NO: 11)
PLYKKIIKKLLES
(SEQ ID NO: 12)
LDLQAPLYKKIIKKLLES

In some aspects, the CXCL4 protein is derived from a human CXCL4 sequence that is at least 85%, at least 90%, at least 95%, at least 96%, at least 97%, at least 98 or at least 99% identical to SEQ ID NO: 1. In some examples, the CXCL4 peptide is no more than 30 amino acids in length, such as a biologically active fragment of CXCL4 that is 12 to 30, 15 to 25, or 19 to 23 amino acids in length. In specific examples, the CXCL4 peptide is a human CXCL4 fragment or variant that is 19, 20, 21, 22 or 23 amino acids in length. In particular examples, the amino acid sequence of the CXCL4 peptide is at least at least 85%, at least 90%, at least 95%, at least 96%, at least 97%, at least 98 or at least 99% identical to SEQ ID NO: 10, SEQ ID NO: 11 or SEQ ID NO: 12. In specific non-limiting examples, the amino acid sequence of the CXCL4 peptide comprises or consists of SEQ ID NO: 10, SEQ ID NO: 11 or SEQ ID NO: 12.

In other particular aspects, the CXCR3 peptide is a fragment or variant of CXCL9. Sequences for CXCL9 proteins from a variety of different species are publicly accessible, such as through the GENBANK™ database. For example, CXCL9 sequences are known for at least the following species: human (see GENBANK™ Gene ID 6373), mouse (Gene ID 56066), rat (Gene ID 305236), chimpanzee (Gene ID 739195), cow (Gene ID 516104), pig (Gene ID 100169744) and macaque (Gene ID 574372).

In some aspects of the compositions and methods disclosed herein, the CXCL9 protein is human CXCL9, or a biologically active fragment thereof. An exemplary CXCL9 protein sequence is provided below.

Human CXCL9 protein
(GENBANK ™ Accession No. NP_002407.1):
(SEQ ID NO: 2)
MKKSGVLFLLGIILLVLIGVQGTPVVRKGRCSCISTNQGTIHL
QSLKDLKQFAPSPSCEKIEIIATLKNGVQTCLNPDSADVKELI
KKWEKQVSQKKKQKNGKKHQKKKVLKVRKSQRSRQKKTT

In some aspects, the CXCL9 peptide is derived from a human CXCL9 sequence that is at least 85%, at least 90%, at least 95%, at least 96%, at least 97%, at least 98 or at least 99% identical to SEQ ID NO: 2. In some examples, the CXCL9 peptide is no more than 30 amino acids in length, such as a biologically active fragment of CXCL9 that is 12 to 30, 15 to 25, or 19 to 23 amino acids in length. In specific examples, the CXCL9 peptide is a human CXCL9 fragment or variant that is 19, 20, 21, 22 or 23 amino acids in length.

In other particular aspects, the CXCR3 peptide is a fragment or variant of CXCL11. Sequences for CXCL11 proteins from a variety of different species are publicly accessible, such as through the GENBANK™ database. For example, CXCL11 sequences are known for at least the following species: human (see GENBANK™ Gene ID 6373), mouse (Gene ID 56066), rat (Gene ID 305236), chimpanzee (Gene ID 739195), cow (Gene ID 516104), pig (Gene ID 100169744) and macaque (Gene ID 574372).

In some aspects of the compositions and methods disclosed herein, the CXCL11 protein is human CXCL11, or a biologically active fragment thereof. An exemplary CXCL11 protein sequence is provided below.

Human CXCL11 protein
(GENBANK ™ Accession No. NP_005400.1):
(SEQ ID NO: 4)
MSVKGMAIALAVILCATVVQGFPMFKRGRCLCIGPGVKAVKV
ADIEKASIMYPSNNCDKIEVIITLKENKGQRCLNPKSKQARL
IIKKVERKNF

In some aspects, the CXCL11 peptide is derived from a human CXCL11 sequence that is at least 85%, at least 90%, at least 95%, at least 96%, at least 97%, at least 98 or at least 99% identical to SEQ ID NO: 4. In some examples, the CXCL11 peptide is no more than 30 amino acids in length, such as a biologically active fragment of CXCL11 that is 12 to 30, 15 to 25, or 19 to 23 amino acids in length. In specific examples, the CXCL11 peptide is a human CXCL11 fragment or variant that is 19, 20, 21, 22 or 23 amino acids in length.

### VI. Summary of Aspects

Aspect 1. A composition, comprising one or more C-X-C chemokine receptor 3 (CXCR3)-activating peptides in a controlled release formulation for ophthalmic administration, wherein the controlled release formulation comprises a coacervate.

Aspect 2. The composition of aspect 1, wherein the coacervate comprises poly(ethylene arginyl aspartate diglyceride) (PEAD) and heparin.

Aspect 3. The composition of aspect 1 or aspect 2, wherein the one or more CXCR3-activating peptides comprise a biologically active fragment or variant of a C-X-C chemokine ligand 4 (CXCL4), CXCL9, CXCL10 or CXCL11 protein.

Aspect 4. The composition of aspect 3, wherein the CXCL4, CXCL9, CXCL10 or CXCL11 protein is a human CXCL4, CXCL9, CXCL10 or CXCL11 protein.

Aspect 5. The composition of any one of aspects 1-4, wherein the one or more peptides are about 12 to about 30 amino acids in length.

Aspect 6. The composition of any one of aspects 1-5, wherein the one or more peptides are about 15 to about 25 amino acids in length.

Aspect 7. The composition of any one of aspects 1-6, wherein the one or more peptides are about 19 to about 23 amino acids in length.

Aspect 8. The composition of any one of aspects 1-7, wherein the amino acid sequences of the one or more peptides are selected from any one of SEQ ID NOs: 5-12.

Aspect 9. The composition of any one of aspects 1-8, comprising a single peptide, wherein the amino acid sequence of the single peptide comprises or consists of

(SEQ ID NO: 5)
PESKAIKNLLKAVHKEMSKRSP
or
(residues 2-21 of SEQ ID NO: 5)
ESKAIKNLLKAVHKEMSKRS.

Aspect 10. The composition of any one of aspects 1-8, comprising at least two peptides, wherein the amino acid sequence of one of the peptides comprises or consists of SEQ ID NO: 5, or residues 2-21 of SEQ ID NO: 5.

Aspect 11. The composition of aspect 9 or aspect 10, wherein the methionine at residue 17 of the peptide of SEQ ID NO: 5 is in the reduced form.

Aspect 12. The composition of aspect 9 or aspect 10, wherein the methionine at residue 17 of the peptide of SEQ ID NO: 5 is in the oxidized form.

Aspect 13. The composition of any one of aspects 1-12, wherein the one or more peptides comprise at least one chemical modification.

Aspect 14. A composition, comprising a C-X-C chemokine receptor 3 (CXCR3)-activating peptide in a controlled release formulation for ophthalmic administration, wherein:
the amino acid sequence of the CXCR3 activating peptide comprises

(SEQ ID NO: 5)
PESKAIKNLLKAVHKEMSKRSP;

the controlled release formulation comprises a coacervate comprising poly(ethylene arginyl aspartate diglyceride) (PEAD) and heparin.

Aspect 15. A method of treating dry eye disease (DED) in a subject, comprising administering to the subject a therapeutically effective amount of the composition of any one of aspects 1-14, thereby treating the DED in the subject.

Aspect 16. The method of aspect 15, wherein the composition is administered topically as an eye drop or eye cream.

Aspect 17. The method of aspect 15, wherein the composition is administered by injection into the conjunctiva.

Aspect 18. The method of any one of aspects 15-17, wherein the composition is administered once per week, twice per week, three times per week, once per day or twice per day.

Aspect 19. The method of aspect 18, wherein the composition is administered daily for about one week, followed by administration 1 to 3 times per week.

Aspect 20. The method of any one of aspects 15-19, wherein the concentration of the CXCR3 activating peptide in the composition is about 5 μg to about 500 μg per 50 μl of the coacervate.

Aspect 21. The method of any one of aspects 15-20, wherein the composition is administered at a volume sufficient to deliver about 10 μg to about 1 mg of the CXCR3 activating peptide.

### EXAMPLES

The following examples are provided to illustrate particular features of certain aspects of the disclosure, but the scope of the claims should not be limited to those features exemplified.

### Example 1: Methods

This example describes the materials and experimental procedures for the studies described in Example 2.

### Materials

The 22-mer CXCL10p peptide (OH-PESKAIKNLLKAVHKEMSKRSP-NH₂; SEQ ID NO: 5) was manufactured by PolyPeptide Inc. (San Diego, CA, USA) and purified to >95% purity as a lyophilized powder. The peptide was delivered in three different formulations. For subconjunctival injection, 3 μg of the peptide was dissolved in 100 μl of sterile PBS for weekly injections. For eye drops, 10 μg of the peptide was dissolved in 50 μl of sterile PBS for twice daily drops (BID). For the slow-release formulation, 10 μg of the peptide was dissolved in 50 μl of coacervate for BID inoculation. The control was 50 μl of sterile PBS applied BID. The coacervate is a heparin and PEAD (1:3.6) mixture with each component at a 10 mg/mL concentration in water. The CXCL10p peptide was added at a 1:10 dilution in PBS to the final concentration.

### Study Design

The environmental dry eye model was used to test the CXCL10p peptide. Female New Zealand white rabbits (8 per group) were used at 125-175 days old (weighing 3-5 kg) at the time of induction. For induction of dry eye, all animals were dosed with atropine sulfate (1%) in both eyes (OU) twice daily (BID) for 2-3 weeks prior to test article administration to induce KCS. In addition, humidity in the animals' housing room was maintained at ~20% throughout the study to promote eye dryness. Non-responder animals were removed from the study at the end of the induction phase. This procedure was continued for all animals remaining on study until study completion.

The study was conducted as outlined in Table 1. The study was carried out in two staggers with half the rabbits in each group (4) being treated in each stagger. The induction and maintenance of desiccation-induced dry eye led to a >30% reduction in Schirmer Tear Test (STT) time, >50% reduction in Tear BreakUp Time (TBUT). Corneal haze was noted in all 64 rabbit eyes at the end of the three-week induction period and was noted in all eyes in the control group at the end of the study.

TABLE 1
Study design

| | | | Route & | Dose Volume | |
|---|---|---|---|---|---|
| Group | N | Treatment | Regimen | (μL/eye) | Slit Lamp Exams |
| 1 | 8 | Control | Topical BID | 50 μL | Baseline (prior to |
| | | | starting on | sterile PBS | induction), once |
| 2 | 8 | Topical Drops | Day 1 (OU) | 50 μL | during induction but |
| | | | | 10 μg | prior to TA admin |
| | | | | CXCL10p | and once weekly |
| 3 | 8 | Topical | | 50 μL | after Day 1 until |
| | | Coacervate | | 10 μg | completion of |
| | | | | CXCL10p | treatment phase |
| 4 | 8 | Sub- | Single | 100 μL | |
| | | Conjunctival | subconjunctival | 3 μg | |
| | | Injection | injection (OU) | CXCL10p | |
| | | | on Days 1, 7, | | |
| | | | and 14 | | |

| | Fluorescein TBUT; | | | | |
|---|---|---|---|---|---|
| | Slit Lamp Photos; | | Conjunctival | | |
| | Fluorescein | Schirmer Tear | Impression | | Tissue |
| Group | Staining | Test | Cytology | Termination | Collection |
| 1 | Prescreen, Baseline | Prescreen, twice | Baseline*, peak | Day 21 | Whole |
| 2 | (prior to TA | weekly (during | of induction*, | | Globes (OU) |
| 3 | administration), and | induction)**, | and prior to | | in 10% |
| 4 | twice weekly after | Baseline (prior | termination | | buffered |
| | Day 1 until the | to TA | | | formalin |
| | completion of | administration) | | | |
| | treatment phase | and after Day 1 | | | |
| | | until the | | | |
| | | completion of | | | |
| | | treatment phase | | | |

BID: twice daily;
OU: both eyes;
TBUT: tear breakup test;
NA: not applicable;
KCS: Keratoconjunctivitis Sicca.
*Performed prior to induction phase.
**Performed during induction to track KCS progress.

### TNFα-Mediated Inflammation

Primary human conjunctival epithelial (HCjE) cells were seeded at 40,000 cells per well in a 96 well flat bottom plate. After overnight adhesion in Keratinocyte-Serum Free Media (cK-SFM), the cells were washed and exposed to starvation media (SFM) for a further 24 hours. At this time the cells were exposed to TNFα (5 ng/ml) in the presence or absence of CXCL10p peptide (200 ng/ml). After 4 hours, the cells were harvested and tested for TNFα mRNA levels with GAPDH mRNA as the control for equalization.

### Goblet Cell Studies

HCjE cells were maintained in complete keratinocyte serum free medium (KSFM; Invitrogen, Carlsbad, CA) in petri dishes coated with type 1 collagen. When the HCjE cells reached 50% confluence, the culture medium was replaced with growth medium (1:1 DMEM/F12:complete KSFM). When cells reached 100% confluence, the cells were fed with stratification medium (DMEM/F12+10% FBS) with or without the CXCL10p peptide (either 200 ng/ml or 2000 ng/ml). Control cells were fed with the medium supplemented with an equivalent volume of the vehicle (PBS) in which the peptide was dissolved. This medium with or without peptide was replaced every other day for 14 days, after which the cells were either stained with periodic acid-Schiff's (PAS) reagent following the standard protocol, or subjected to total RNA isolation, and qRT-PCR for MUC5AC transcripts with TATA-box binding protein (TBP) as the endogenous control.

### Example 2: a CXCR3-Activating Peptide Increases Tear Break-Up Time (TBUT) in a Rabbit model of environmental dry eye

Treated and control rabbits were evaluated by Schirmer tear test (STT) and TBUT times. For STT, the baseline values of 12-15 mm were reduced minimally to 9-12 mm after induction and tended to drift lower during treatment to 8-11 mm, regardless of the delivery mode (Table 2). Thus, the amount of aqueous tear was uncorrected by the CXCR3 activating peptide.

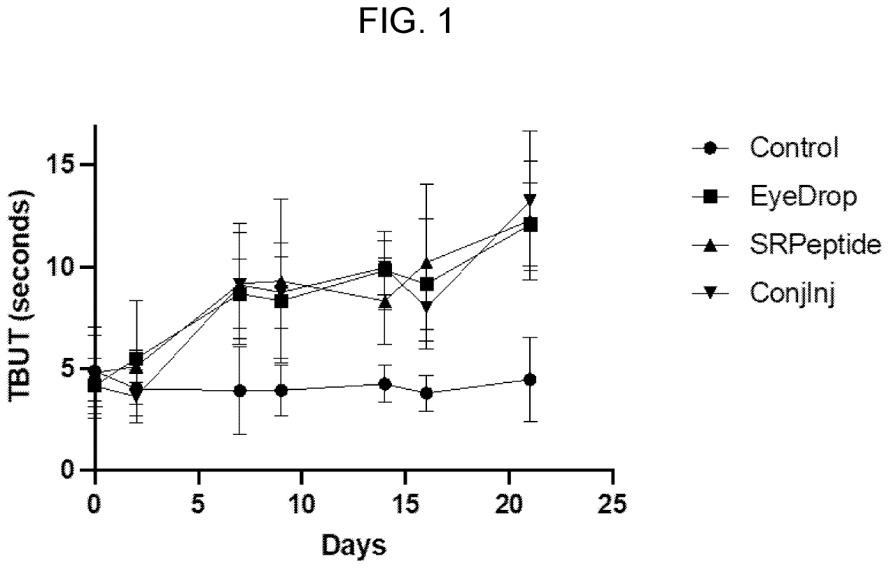

However, TBUT was altered more significantly by the induction of dry eye and subsequent treatment with CXCR3 activating peptide. Initial TBUT times of 8-9 s were reduced to half (3.5-5 s) after induction and prior to treatment (Table 2). TBUT remained low in the saline treated eyes but rebounded to baseline in the first week of treatment for all three treated groups (FIG. 1). In all three treatment groups, the TBUT exceeded baseline by the end of week 3 (Table 3), while the eye drop group trended higher than baseline but did not reach statistical significance (FIG. 1). This increase in TBUT was also noted when analyzing each stagger separately. The increase in TBUT combined with no effect on STT suggested that either the mucin or lipid layer was altered.

TABLE 2
STT values prior to induction, prior to treatment and after treatment

| | Day −21 | | Day 0 | | Day 21 | | |
|---|---|---|---|---|---|---|---|
| Treatment | OD | OS | OD | OS | OD | OD | P Value |
| Saline control | 15.5 ± 3.2 | 13.1 ± 2.9 | 12.1 ± 3.6 | 9.0 ± 2.9 | 8.4 ± 2.2 | 8.3 ± 3.0 | n.s. |
| Eyedrop | 14.6 ± 5.6 | 14.0 ± 2.8 | 11.3 ± 3.3 | 9.9 ± 3.7 | 9.9 ± 3.9 | 7.9 ± 2.0 | n.s. |
| SR Peptide | 14.6 ± 3.3 | 13.0 ± 4.1 | 9.8 ± 3.4 | 9.3 ± 3.2 | 9.4 ± 2.9 | 8.4 ± 1.8 | n.s. |
| Subconjunctival | 12.6 ± 2.8 | 12.5 ± 2.7 | 10.3 ± 2.5 | 8.8 ± 2.7 | 11.0 ± 1.9 | 9.8 ± 2.0 | n.s. |
| injection | | | | | | | |

OD—right eye;
OS—left eye
Shown are mean ± s.d. for 8 eyes in each column.
P values are day 21 versus at induction (Day 0).
The saline control was significantly reduced from prior to induction but unchanged from prior to treatment.

TABLE 3
TBUT values prior to induction, prior to treatment and after treatment

| | Day −21 | | Day 0 | | Day 21 | | |
|---|---|---|---|---|---|---|---|
| Treatment | OD | OS | OD | OS | OD | OD | P Value |
| Saline Control | 8.6 ± 2.7 | 8.3 ± 1.7 | 4.9 ± 2.2 | 4.7 ± 1.2 | 4.8 ± 1.7 | 4.1 ± 2.1 | |
| Eyedrop | 8.7 ± 2.0 | 8.4 ± 1.4 | 4.0 ± 0.6 | 4.7 ± 1.3 | 11.3 ± 4.2 | 10.9 ± 4.7 | >0.01 |
| SR Peptide | 7.9 ± 2.0 | 7.9 ± 1.7 | 4.7 ± 1.8 | 4.7 ± 1.6 | 13.3 ± 4.1 | 12.7 ± 5.1 | >0.01 |
| Subconjunctival | 9.0 ± 3.9 | 8.5 ± 2.3 | 3.7 ± 0.7 | 4.9 ± 2.3 | 13.2 ± 5.8 | 12.7 ± 5.3 | >0.01 |
| injection | | | | | | | |

OD: right eye;
OS: left eye
Shown are mean ± s.d. for 8 eyes in each column.
P values are day 21 versus prior to induction (Day −21).
The saline control was significantly reduced from prior to induction but unchanged from prior to treatment.

The increase in the TBUT combined with no effect on STT suggested that either the mucin or lipid layer was altered, in accordance with earlier findings on an increase in goblet cells after toxic (mitomycin-C) injury (Swogger et al., Clin Exp Ophthalmol 49:60-69, 2021). Thus, it was investigated whether the CXCR3 activating peptide altered the goblet cells. Histopathological examination of enucleated globes at the end of the study (day 21 of treatment) found that goblet cell density was similarly high in all treatment and control groups.

However, the increase in mucin production did appear to alleviate the desiccation-induced corneal pathology. In all groups, all 16 eyes displayed corneal pathology at the end of the three-week induction period. At the end of the three-week test article administration, the control group still had corneal pathology in all 16 eyes. However, the number of affected eyes were reduced after treatment with eyedrops (4 of 16 eyes showing pathology), slow-release gel formulation (2 of 16) and subconjunctival injection (6 of 16). All treatments were statistically different from the saline-treated control (at P<0.05).

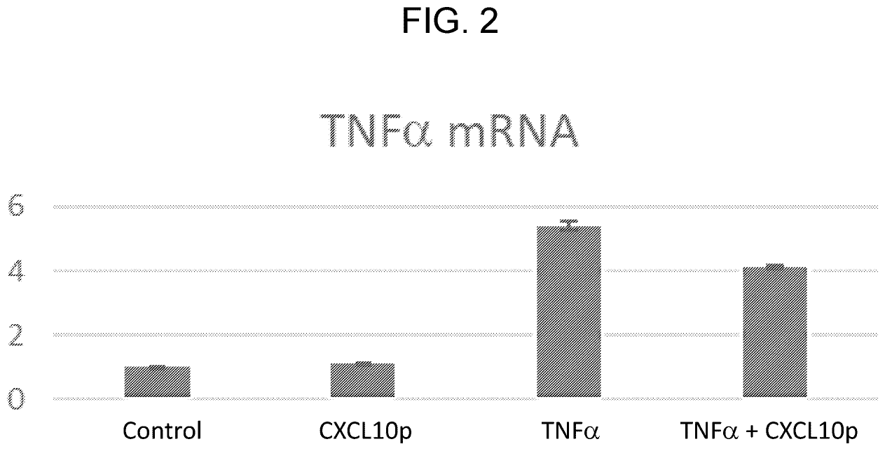

An initial in vitro study to discern the underlying mechanism of action in addition to the goblet cell number and functioning, focused on the anti-inflammatory nature of CXCR3 signaling (Wells, Int J Biochem Cell Biol 152:106311, 2022). The inflammatory potential of conjunctival epithelial cells that are involved during flares in DED were examined (Perez et al., Exp Eye Res 201:108294, 2020). As TNFα is central to the propagation of the inflammation that damages the goblet cells, HCjE were challenged with TNFα and the subsequent de novo production of TNFα mRNA was evaluated in these cells (FIG. 2). Concurrent exposure to peptide dampened the TNFα mRNA levels in a reproducible and statistically significant manner.

To further examine the effects of the CXCR3 activating peptide on goblet cell functioning in vitro, stratified HCjE cells are treated with 200 ng/ml CXCR3 activating peptide, 2000 ng/ml CXCR3 activating peptide, or vehicle. Cells are then stained with periodic acid-Schiff's (PAS) reagent. It is expected that compared to control vehicle-treated HCjE cells, cells treated with either 200 ng/ml or 2000 ng/ml of the CXCR3 activating peptide will show more intense staining with PAS reagent, indicating increased proteoglycan content in these cells, as well as an increase in the frequency of intense magenta stained cell clusters indicative of goblet cell clusters.

Control and CXCR3 activating peptide-treated HCjE cells are also examined for expression of MUC5AC transcripts by qRT-PCR. It is expected that HCjE cells treated with 200 ng/ml or 2000 ng/ml of the CXCR3 activating peptide will have increased levels of MUC5AC transcripts relative to animals treated with vehicle only, which indicates that the CXCR3 activating peptide promotes differentiation of HCjE cells towards goblet cell fate.

It will be apparent that the precise details of the methods or compositions described may be varied or modified without departing from the spirit of the described aspects of the disclosure. We claim all such modifications and variations that fall within the scope and spirit of the claims below.

1. A composition, comprising one or more C-X-C chemokine receptor 3 (CXCR3)-activating peptides in a controlled release formulation for ophthalmic administration, wherein the controlled release formulation comprises a coacervate.

2. The composition of claim 1, wherein the coacervate comprises poly(ethylene arginyl aspartate diglyceride) (PEAD) and heparin.

3. The composition of claim 1, wherein the one or more CXCR3-activating peptides comprise a biologically active fragment or variant of a C—X—C chemokine ligand 10 (CXCL10), CXCL4, CXCL9, or CXCL11 protein.

4. The composition of claim 3, wherein the CXCL10, CXCL4, CXCL9, or CXCL11 protein is a human CXCL10, CXCL4, CXCL9, or CXCL11 protein.

5. The composition of claim 1, wherein the one or more peptides are about 12 to about 30 amino acids in length.

6. The composition of claim 1, wherein the one or more peptides are about 15 to about 25 amino acids in length.

7. The composition of claim 1, wherein the one or more peptides are about 19 to about 23 amino acids in length.

8. The composition of claim 1, wherein the amino acid sequences of the one or more peptides are selected from any one of SEQ ID NOs: 5-12.

9. The composition of claim 1, comprising a single peptide, wherein the amino acid sequence of the single peptide comprises or consists of PESKAIKNLLKAVHKEMSKRSP (SEQ ID NO: 5) or ESKAIKNLLKAVHKEMSKRS (residues 2-21 of SEQ ID NO: 5).

10. The composition of claim 1, comprising at least two peptides, wherein the amino acid sequence of one of the peptides comprises or consists of SEQ ID NO: 5, or residues 2-21 of SEQ ID NO: 5.

11. The composition of claim 9, wherein the methionine at residue 17 of the peptide of SEQ ID NO: 5 is in the reduced form.

12. The composition of claim 9, wherein the methionine at residue 17 of the peptide of SEQ ID NO: 5 is in the oxidized form.

13. The composition of claim 1, wherein the one or more peptides comprise at least one chemical modification.

14. A composition, comprising a C-X-C chemokine receptor 3 (CXCR3)-activating peptide in a controlled release formulation for ophthalmic administration, wherein:
the amino acid sequence of the CXCR3 activating peptide comprises
(SEQ ID NO: 5)
PESKAIKNLLKAVHKEMSKRSP;
and
the controlled release formulation comprises a coacervate comprising poly(ethylene arginyl aspartate diglyceride) (PEAD) and heparin.

15. A method of treating dry eye disease (DED) in a subject, comprising administering to the subject a therapeutically effective amount of the composition of claim 1, thereby treating the DED in the subject.

16. The method of claim 15, wherein the composition is administered topically as an eye drop or eye cream.

17. The method of claim 15, wherein the composition is administered by injection into the conjunctiva.

18. The method of claim 15, wherein the composition is administered once per week, twice per week, three times per week, once per day or twice per day.

19. The method of claim 18, wherein the composition is administered daily for about one week, followed by administration 1 to 3 times per week.

20. The method of claim 15, wherein:
the concentration of the CXCR3 activating peptide in the composition is about 5 μg to about 500 μg per 50 μl of the coacervate; and/or
the composition is administered at a volume sufficient to deliver about 10 μg to about 1 mg of the CXCR3 activating peptide.


SEQUENCE LISTING (12 sequences)

SEQ ID NO: 1 - protein, 101 aa, Homo sapiens
MSSAAGFCASRPGLLFLGLLLLPLVVAFASAEAEEDGDLQCLCVKTTSQVRPRHITSLEVIKAGPHCPTAQLIATLKNGRKICLDLQAPLYKKIIKKLLES

SEQ ID NO: 2 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 3 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 4 - protein, 101 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNFSEQIDNO

SEQ ID NO: 5 - protein, 22 aa, synthetic construct
PESKAIKNLLKAVHKEMSKRSP

SEQ ID NO: 6 - protein, 22 aa, synthetic construct
PESKAIKNLLKAVSKEMSKRSP

SEQ ID NO: 7 - protein, 21 aa, synthetic construct
ESKAIKNLLKAVSKERSKRSP

SEQ ID NO: 8 - protein, 22 aa, synthetic construct
PESKAIKNLLKAVSKERSKRSP

SEQ ID NO: 9 - protein, 21 aa, synthetic construct
ESKAIKNLLKAVSKEMSKRSP

SEQ ID NO: 10 - protein, 29 aa, synthetic construct
DLQCLCVKTTSQVRPRHITSLEVIKAGPH

SEQ ID NO: 11 - protein, 13 aa, synthetic construct
PLYKKIIKKLLES

SEQ ID NO: 12 - protein, 18 aa, synthetic construct
LDLQAPLYKKIIKKLLES